(19) United States
(12) Patent Application Publication
(10) Pub. No.: US 2014/0273197 A1
(43) Pub. Date: Sep. 18, 2014

(54) VIBRATABLE CULTURE APPARATUS FOR PROVIDING PLANT CELLS WITH GROWTH ENVIRONMENT

(71) Applicant: CASHIDO CORPORATION, Miaoli County (TW)

(72) Inventors: SHIH-CHANG CHEN, HSINCHU COUNTY (TW); CHUN-LUNG CHIU, HSINCHU COUNTY (TW); CHIH-HSIANG CHEN, HSINCHU COUNTY (TW); YUAN-PENG LEE, MIAOLI COUNTY (TW); PO-HUNG LAI, MIAOLI COUNTY (TW); I-CHIAO LIN, TAIPEI CITY (TW); PUNG-LING HUANG, MIAOLI COUNTY (TW); YI-YIN DO, MIAOLI COUNTY (TW); CHENG-KU LIN, MIAOLI COUNTY (TW)

(73) Assignee: CASHIDO CORPORATION, MIAOLI COUNTY (TW)

(21) Appl. No.: 13/826,202

(22) Filed: Mar. 14, 2013

(51) Int. Cl. C12M 1/00

(52) U.S. Cl. CPC C12M 21/02
USPC 435/292.1

(57) ABSTRACT

A vibratable culture apparatus includes a culture device and a vibrating device having a driving module and a vibrating module. The vibrating module has a working platform connected to the driving module, a plurality of lighting units installed on the working platform, and a plurality of positioning units. The working platform is vibratable in a plane by the driving module. Each lighting unit has a first LED set used and a second LED set. The positioning units are installed on the working platform and respectively arranged adjacent to the lighting units. The culture device is detachably disposed on the working platform and restricted by at least partial the positioning units for maintaining the relative position between the culture device and the working platform. The culture device is arranged above at least partial the lighting units that used for emitting light to penetrate into the culture device.

### BACKGROUND OF THE INVENTION

1. Field of the Invention

The instant disclosure relates to a culture apparatus; more particular, to a vibratable culture apparatus and a vibrating device, which are used for providing a plurality of plant cells with a growth environment.

2. Description of Related Art

The plant cell culture process is usually implemented with vibration of the vibrating device for activating the plant cells to improve the success rate of cultivation.

However, the conventional culture device (e.g., the glass bottle) in the plant cell culture process cannot provide sufficient light to the plant cells. For example, the plant cells, which are arranged in the glass bottle and cultured by lighting, rely on the external lighting device for emitting light from the top of the glass bottle into the glass bottle, so that the plant cells absorb the light. The opening of the glass bottle must be sealed or stuffed, so that partial of the light is stopped before emitting into the glass bottle. Thus, the conventional device cannot provide sufficient light for the plant cells during the plant cell culture process.

To achieve the abovementioned improvement, the inventors strive via industrial experience and academic research to present the instant disclosure, which can provide additional improvement as mentioned above.

### SUMMARY OF THE INVENTION

One embodiment of the instant disclosure provides a vibratable culture apparatus and a vibrating device, which are used for providing a plurality of plant cells with a growth environment to promote the growth of the plant cells and increase success rate of the cultivation of the plant cells.

The vibratable culture apparatus for providing the plant cells with the growth environment, comprises: a vibrating device comprising: a driving module; a vibrating module installed on the driving module, the vibrating module comprising: a working platform connected to the driving module, wherein the working platform is vibratable in a plane by the driving module; a plurality of lighting units installed on the working platform, wherein each lighting unit has a first LED set used for emitting a red light and a second LED set used for emitting a blue light, wherein the wavelength range of the red light is approximately 620 nm to 760 nm, the wavelength range of the blue light is approximately 360 nm to 480 nm; and a plurality of positioning units installed on the working platform and respectively arranged adjacent to the lighting units: and a culture device detachably disposed on at least partial the lighting units that used for emitting light to penetrate into the culture device, wherein the culture device is restricted by at least partial the positioning units for maintaining the relative position between the culture device and the working platform.

The vibrating device comprises: a driving module; and a vibrating module installed on the driving module, the vibrating module comprising: a working platform connected to the driving module, wherein the working platform is vibratable in a plane by the driving module; a plurality of lighting units installed on the working platform, wherein each lighting unit has a first LED set used for emitting red light and a second LED set used for emitting blue light, wherein the wavelength range of the red light is approximately 620 nm to 760 nm, the wavelength range of the blue light is approximately 360 nm to 480 nm; and a plurality of positioning units installed on the working platform and respectively arranged adjacent to the lighting units; and a controlling module electrically connected to the lighting units, wherein the light proportion emitted from each lighting unit is adjustable by the controlling module.

Base on the above, the vibratable culture apparatus and the vibrating device of the instant disclosure provide the suitable growth environment for the plant cells, which is arranged on the vibrating device, by installing the lighting units on the working platform and vibrating the working platform.

### BRIEF DESCRIPTION OF THE DRAWINGS

FIG. 1 is a perspective view of a vibratable culture apparatus of the instant disclosure, which is used for providing a plurality of plant cells with a growth environment:

FIG. 2 is a block diagram of a vibrating device of the vibratable culture apparatus of the instant disclosure;

FIG. 3 is a using state view of the vibratable culture apparatus of the instant disclosure.

### DETAILED DESCRIPTION OF THE PREFERRED EMBODIMENTS

In order to further appreciate the characteristics and technical contents of the instant disclosure, references are hereunder made to the detailed descriptions and appended drawings in connection with the instant disclosure. However, the appended drawings are merely shown for exemplary purposes, rather than being used to restrict the scope of the instant disclosure.

Please refer to FIGS. 1 to 3, which show an embodiment of the instant disclosure. The embodiment provides a vibratable culture apparatus 100 for providing a plurality of plant cells 200 with a growth environment. The vibratable culture apparatus 100 disposed on a working surface (e.g., ground) has a vibrating device 1 and a culture device 2 disposed on the vibrating device 1.

Moreover, the vibratable culture apparatus 100 has a static state or a vibrating state by operating the vibrating device 1. The following description states the structural features of the vibrating device 1 and the culture device 2, wherein each element is stated firstly, and the relationships of the corresponding elements are stated later. After that, the description states the operation process of the vibrating device 1 and the culture device 2.

The vibrating device 1 has a driving module 11 and a vibrating module 12 installed on the driving module 11. The driving module 11 can be a motor or the other driving piece, but not limited thereto.

Moreover, the installed method between the driving module 11 and the vibrating module 12 is directly connected or indirectly connected via a transmission mechanism (e.g., gear set, connecting rod, or track). The driving module 11 is electrically connected to an external power (e.g., conventional power or power generator) for providing the operating energy to the driving module 11.

The vibrating module 12 has a working platform 121, a plurality of lighting units 122, a controlling unit 123, and a plurality of positioning units 124. The lighting units 122 and the positioning units 124 are installed on the working platform 121, and the controlling unit 123 is electrically connected to the lighting units 122.

Specifically, the working platform 121 has a plate-like shape, and the working platform 121 has a plurality of receiving troughs 1212 concavedly formed on an outer surface 1211 thereof. The amount of the receiving troughs 1212 is equal to the amount of the lighting units 122. The depth of each receiving trough 1212 is suitable for receiving one lighting unit 122, wherein the lighting unit 122 does not protrude over the outer surface 1211 of the working platform 121.

Additionally, the working platform 121 is connected to the driving module 11 by directly connection type or indirectly connection type. The working platform 121 is vibratable in a plane (not labeled) by the driving module 11. In other words, the working platform 121 is situated the vibrating state by the driving module 11.

The vibrating type of the working platform 121 is uninterruptedly moving along a predetermined path (e.g., circular path, ellipse path, or irregular curve) or reciprocating in two opposite directions along the predetermined path, but not limited thereto.

Specifically, the plane in this embodiment is parallel to a water level, that is to say, the outer surface 1211 of the working platform 121 is approximately parallel to the water level, but not limited thereto. For example, in another embodiment, which not shown in figures, the plane and the water level has an acute angle arranged therebetween, that is to say, the outer surface 1211 of the working platform 121 and the water level has the acute angle arranged therebetween.

Each lighting unit 122 has a plurality of LEDs, which are classified according to the light sorts of the LEDs in this embodiment. Thus, each lighting unit 122 has a first LED set 1221 used for emitting a red light, a second LED set 1222 used for emitting a blue light, and a third LED set 1223 used for emitting a green light.

The first LED set 1221, the second LED set 1222, and the third LED set 1223 each has a plurality of LEDs. The wavelength range of the red light is approximately 620 nm to 760 nm, the wavelength range of the blue light is approximately 360 nm to 480 nm, and the wavelength range of the green light is approximately 480 nm to 530 nm.

Moreover, each lighting unit 122 further has a first UV set 1224 used for emitting an ultraviolet A (UVA), a second UV set 1225 used for emitting an ultraviolet B (UVB), and a third UV set 1226 used for emitting an ultraviolet C (UVC). The wavelength range of the UVA is approximately 320 nm to 400 nm, the wavelength range of the UVB is approximately 290 nm to 320 nm, and the wavelength range of the UVC is approximately 200 nm to 290 nm.

Incidentally, the above six light sets 1221˜1226 of each lighting unit 122 has a specific proportion. Furthermore, the proportion of the above six light sets 1221˜1226 of each lighting unit 122 can be adjusted by the designer's demand.

The lighting units 122 are installed on the working platform 121. Furthermore, the lighting units 122 are respectively received in the receiving troughs 1212 and do not protrude over the outer surface 1211 of the working platform 121.

The controlling module 123 is electrically connected to the lighting units 122, and the light proportion emitted from each lighting unit 122 is adjustable by the controlling module 123. In other words, the controlling module 123 is provided for the user to control the light proportion emitted from each lighting unit 122. For example, the light proportion of the first LED set 1221, the second LED set 1222, and the third LED set 1223 is 7:2:1, but not limited thereto.

The controlling module 123 further has a timer 1232 electrically connected to the first UV set 1224, the second UV set 1225, and the third UV set 1226 of each lighting unit 122 for controlling the lighting time of each UV set 1224˜1226.

Moreover, the controlling module 123 is installed on the working platform 121 or disposed outside the working platform 121. For example, please refer to FIG. 1, the controlling module 123 has a plurality of controllers 1231, and the amount of the controllers 1231 is equal to the amount of the lighting units 122. The controllers 1231 are installed on the working platform 121 and respectively electrically connected to the lighting units 122. The light proportion of each lighting unit 122 is independently adjusted by each corresponding controller 1231.

Or, the controlling module 123 is an one piece structure (not shown), which does not has the controllers 1231. Thus, the light proportion of each lighting unit 122 is adjusted by the circuit design of the controlling module 123.

Each positioning unit 124 has a plurality of positioning pillars 1241 installed on the outer surface 1211 of the working platform 121. The positioning units 124 are respectively arranged adjacent to the lighting units 122.

Specifically, a portion of the outer surface 1211, which is connected to the positioning pillars 1241 of each positioning unit 124, is surrounding each lighting unit 122, so that each lighting unit 122 and the adjacent positioning pillars 1241 surroundingly define a receiving space 125. Besides, the positioning unit 124 in this embodiment takes the positioning pillars 1241 for example, but not limited thereto.

The culture device 2 has at least one transparent conical culture bottle 21 (e.g., conical flask) and at least one cap 22 (e.g., rubber bung), wherein the amount of the culture bottle 21 is equal to the amount of the cap 22. Moreover, the amount of the culture bottle 21 can be adjusted by user, that is to say, the amount of the culture bottle 21 does not need to equal to the amount of the lighting units 122.

Each culture bottle 21 has a bottom surface 211 and a rim 212, and the area of the bottom surface 211 is larger than the area surrounded by the rim 212. The cap 22 is used for inserting into the rim 212, and the culture bottle 21 and the cap 22 surroundingly define a closed accommodating space 23.

The bottom surface 211 of the culture bottle 21 is disposed on the lighting unit 122 that used for emitting light to penetrate into the accommodating space 23 of the culture device 21. The culture bottle 21 contacts the adjacent positioning pillars 1241 and arranged in the receiving space 125 surroundingly defined by the adjacent positioning pillars 1241, so that the culture bottle 21 is restricted by the positioning pillars 1241 for maintaining the relative position between the culture bottle 21 and the working platform 121.

The structural features of the vibrating device 1 and the culture device 2 have been stated in the above description, and the following description states the operation process of the vibrating device 1 and the culture device 2.

Please refer to FIGS. 2 and 3. Each culture bottle 21 receives the plant cells 200 and a tissue fluid 300, and the cap 22 inserts into the rim 212 of the culture bottle 21.

The light proportion of lighting unit 122 is adjusted by the controlling module 123 according to the growth condition of the plant cells 200 arranged in the culture bottle 21. Moreover, the user can further control at least one of the first UV set 1224, the second UV set 1225, and the third UV set 1226 to emit UV light. The user can also control the lighting time of the UV light by the timer 1232 for preventing the plant cells 200 from damage or even death.

And then, the culture bottle 21, which receives the plant cells 200 and the tissue fluid 300, is disposed on the lighting unit 122 adjusted the light proportion already. The lighting unit 122 emits light to pass through the bottom surface 211 of the bottle 21 for illuminating the plant cells 200 and the tissue fluid 300.

Moreover, operating the driving module 11 to vibrate the working platform 121, so that the working platform 121 and the culture bottle 21 disposed thereon are moving along the predetermined path for situating the vibrating state.

Thus, the vibratable culture apparatus 100 provides the growth environment (vibrating and lighting), which is suitable for growing the plant cells 200 arranged in each culture bottle 21.

Specifically, the vibratable culture apparatus 100 in this embodiment is used for receiving one kind of the plant cell for example, but not limited thereto. That is to say, the vibratable culture apparatus 100 in use can be used for receiving any kind of the plant cells.

Base on the above, the vibratable culture apparatus of the instant disclosure provides the culture bottles to dispose thereon at the same time by installing the lighting units on the working platform. The working platform situates the vibrating state by the driving module for promoting the growth of the plant cells arranged in the culture bottles and improving the success rate of cultivation of the plant cells arranged in the culture bottles.

Moreover, according to the kind of the plant cells, the lighting units of the vibrating device has different light sets, such as the LED sets respectively for emitting light with different colors, or the UV sets respectively for emitting UV light with different wavelengths.

The descriptions illustrated supra set forth simply the preferred embodiments of the instant disclosure; however, the characteristics of the instant disclosure are by no means restricted thereto. All changes, alternations, or modifications conveniently considered by those skilled in the art are deemed to be encompassed within the scope of the instant disclosure delineated by the following claims.

What is claimed is:

1. A vibratable culture apparatus for providing a plurality of plant cells with a growth environment, comprising:
a vibrating device comprising:
a driving module;
a vibrating module installed on the driving module, the vibrating module comprising:
a working platform connected to the driving module, wherein the working platform is vibratable in a plane by the driving module;
a plurality of lighting units installed on the working platform, wherein each lighting unit has a first LED set used for emitting a red light and a second LED set used for emitting a blue light, wherein the wavelength range of the red light is approximately 620 nm to 760 nm, the wavelength range of the blue light is approximately 360 nm to 480 nm; and
a plurality of positioning units installed on the working platform and respectively arranged adjacent to the lighting units; and
a culture device detachably disposed on at least partial the lighting units that used for emitting light to penetrate into the culture device, wherein the culture device is restricted by at least partial the positioning units for maintaining the relative position between the culture device and the working platform.

2. The vibratable culture apparatus as claimed in claim 1, wherein the vibrating device further comprises a controlling module electrically connected to the lighting units, and wherein the light proportion emitted from each lighting unit is adjustable by the controlling module.

3. The vibratable culture apparatus as claimed in claim 2, wherein each positioning unit has a plurality of positioning pillars disposed on the working platform and surrounding each lighting unit, the culture device has at least one transparent conical culture bottle disposed above at least one of the lighting units, and the culture bottle contacts the adjacent positioning pillars and arranged in a space surroundingly defined by the adjacent positioning pillars.

4. The vibratable culture apparatus as claimed in claim 2, wherein the working platform has a plurality of receiving troughs concavedly formed on a surface thereof, and wherein the lighting units are respectively arranged in the receiving troughs.

5. The vibratable culture apparatus as claimed in claim 2, wherein the plane is parallel to a water level.

6. The vibratable culture apparatus as claimed in claim 1, wherein each lighting unit further has a third LED set used for emitting a green light, and wherein the wavelength range of the green light is approximately 480 nm to 530 nm.

7. The vibratable culture apparatus as claimed in claim 6, wherein each lighting unit further has a first UV set used for emitting an ultraviolet A (UVA), and wherein the wavelength range of the UVA is approximately 320 nm to 400 nm.

8. The vibratable culture apparatus as claimed in claim 7, wherein each lighting unit further has a second UV set used for emitting an ultraviolet B (UVB), and wherein the wavelength range of the UVB is approximately 290 nm to 320 nm.

9. The vibratable culture apparatus as claimed in claim 8, wherein each lighting unit further has a third UV set used for emitting an ultraviolet C (UVC), and wherein the wavelength range of the UVC is approximately 200 nm to 290 nm.

10. A vibrating device, comprising:
a driving module; and
a vibrating module installed on the driving module, the vibrating module comprising:
a working platform connected to the driving module, wherein the working platform is vibratable in a plane by the driving module;
a plurality of lighting units installed on the working platform, wherein each lighting unit has a first LED set used for emitting red light and a second LED set used for emitting blue light, wherein the wavelength range of the red light is approximately 620 nm to 760 nm, the wavelength range of the blue light is approximately 360 nm to 480 nm; and
a plurality of positioning units installed on the working platform and respectively arranged adjacent to the lighting units; and
a controlling module electrically connected to the lighting units, wherein the light proportion emitted from each lighting unit is adjustable by the controlling module.

11. The vibrating device as claimed in claim 10, wherein each positioning unit has a plurality of positioning pillars disposed on the working platform and arranged surrounding each lighting unit.

12. The vibrating device as claimed in claim 10, wherein the working platform has a plurality of receiving troughs concavedly formed on a surface thereof, and wherein the lighting units are respectively arranged in the receiving troughs.

13. The vibrating device as claimed in claim 10, wherein the plane is parallel to a water level.

14. The vibrating device as claimed in claim 10, wherein each lighting unit further has a third LED set used for emitting a green light, and wherein the wavelength range of the green light is approximately 480 nm to 530 nm.

15. The vibrating device as claimed in claim 14, wherein each lighting unit further has a first UV set used for emitting an ultraviolet A (UVA), a second UV set used for emitting an ultraviolet B (UVB), and a third UV set used for emitting an ultraviolet C (UVC), and wherein the wavelength range of the UVA is approximately 320 nm to 400 nm, the wavelength range of the UVB is approximately 290 nm to 320 nm, and the wavelength range of the UVC is approximately 200 nm to 290 nm.
